(19) United States
(12) Patent Application Publication
(10) Pub. No.: US 2021/0047615 A1
(43) Pub. Date: Feb. 18, 2021

(54) COMPOSITION FOR EMBRYONIC DEVELOPMENT, COMPRISING RAD51 ACTIVATOR, AND METHOD FOR IMPROVING EMBRYONIC DEVELOPMENT RATE USING SAME

(71) Applicant: CHA UNIVERSITY INDUSTRY-ACADEMIC COOPERATION FOUNDATION, Pocheon-si (KR)

(72) Inventors: Dong Ryul LEE, Seoul (KR); Kyung Soon PARK, Seongnam-si (KR); Ah Reum LEE, Seongnam-si (KR)

(73) Assignee: CHA UNIVERSITY INDUSTRY-ACADEMIC COOPERATION FOUNDATION, Pocheon-si (KR)

(21) Appl. No.: 16/964,439

(22) PCT Filed: Jan. 23, 2019
(86) PCT No.: PCT/KR2019/000988

§ 371 Date: Jul. 23, 2020

(30) Foreign Application Priority Data
Jan. 23, 2018 (KR) 10-2018-0008250

(51) Int. Cl. C12N 5/0735; C12N 5/073; A61D 19/04

(52) U.S. Cl. CPC C12N 5/0606; C12N 5/0604; A61B 17/435; C12N 2501/999; A61D 19/04

(57) ABSTRACT

An aspect provides a method of increasing efficiency of somatic cell nuclear transfer using a substance (RS-1) increasing Rad51 activity, and a somatic cell nuclear transfer cell prepared according to the method. Another aspect provides a method of screening for a substance increasing Rad51 activity to increase efficiency of somatic cell nuclear transfer. When the substance increasing Rad51 activity is used, efficiency of somatic cell nuclear transfer may be increased.

### TECHNICAL FIELD

The present disclosure relates to a composition for embryonic development, the composition including a Rad51 activator, and a method of improving an embryonic development rate using the same.

### BACKGROUND ART

The most advantageous method of producing stem cells compatible with a patient's immune system may be a method of producing embryonic stem cells through somatic cell nuclear transfer (SCNT). However, the SCNT technology has very low production efficiency, and the cause of failure has not been clearly investigated. Therefore, identifying the cause of low production efficiency and increasing production efficiency remain a challenge to be solved for commercialization of stem cells by the SCNT.

For homeostasis of a genome, DNA is required to be preserved without damages, among which the most fatal DNA damage is break of DNA double helix. Rad51 is one of proteins involved in the repair of broken DNA double helix. When break of DNA double helix occurs, single-stranded DNA is first formed at the site of break by exonuclease, and the single strand is protected by coating with replication protein A (RPA). Subsequently, the recombinant protein Rad51 replaces RPA to form a filament-shaped complex, searches for homologous chromosomes, etc., finds a homologous nucleotide sequence, and then exchanges the DNA strand to complete homologous recombination repair.

In addition, rad51-stimulatory compound 1 (RS-1) which is a chemical increasing the enzymatic activity of Rad51 is known to improve homologous recombination efficiency by increasing DNA binding activity of Rad51.

### DESCRIPTION OF EMBODIMENTS

### Technical Problem

An aspect provides a composition for embryonic development, the composition including a substance increasing Rad51 activity.

Another aspect provides a method of increasing efficiency of embryonic development, the method including culturing an egg in a medium including the substance increasing Rad51 activity.

### Solution to Problem

An aspect provides a composition for increasing embryo formation or development efficiency of a fertilized egg, the composition including a substance increasing Rad51 activity. Another aspect provides a composition for increasing embryo formation or development efficiency, the composition including a Rad51 activator.

As used herein, the term “embryo formation” means that a zygote becomes a number of cells through cell division, and these cells form an embryo or develops as an embryo through cell division and differentiation.

As used herein, the term “increase of efficiency” refers to blastocyst development of a somatic cell cloned egg or an increase of blastocyst development. The blastocyst refers to a fertilized egg which develops enough to be divided into an inner cell mass, which differentiates into a fetus, and a trophectoderm, which differentiates into a placenta, by forming a blastocyst cavity after a dense morula during repeated division and growth of the fertilized egg. Therefore, the increase of efficiency refers to an increase of efficiency of a somatic cell nuclear transfer, specifically, an increase of embryonic development efficiency of a somatic cell cloned egg, an increase of development of a somatic cell nuclear transfer embryo to a blastocyst stage, an increase of development to a blastocyst stage, an increase of blastocyst production efficiency, an increase of blastocyst yield efficiency, or an increase of a blastocyst formation rate, as compared with a somatic cell nuclear transfer which is performed in the absence of an agent reducing H3K9me3 methylation.

As used herein, the term “Rad51”, which is a gene expressed in eukaryotes, refers to a member of the Rad51 protein family that is involved in repair of double stranded DNA breaks, that is, a DNA corrector. A sequence and location of the gene are known in the art (NCBI Gene ID: 5888, etc.).

The substance increasing Rad51 activity may be, for example, a commercially available compound having a product name of RAD51-stimulatory compound-1 (RS-1), or a substance binding to Rad51 screened according to a method known in the art such as high throughput screening (HTS), etc., or (3-[(benzylamino)sulfonyl]-4-bromo-N-(4-bromophenyl)benzamide), 4-bromo-N-(4-bromophenyl)-3-[[(phenylmethyl)amino]sulfonyl]-benzamide), or a compound of the following Formula 1, or a derivative thereof:

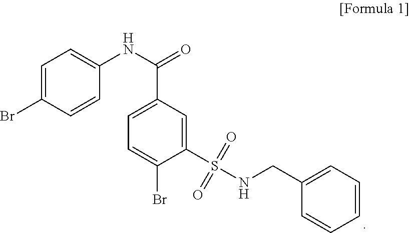 [SMILES: O=C(Nc1ccc(Br)cc1)c1ccc(Br)c(S(=O)(=O)NCc2ccccc2)c1]

The substance increasing Rad51 activity may have a function of maintaining a DNA correction effect, since it increases the activity of Rad51 protein by maintaining binding stability of Rad51 protein having the function of correcting damaged DNA with respect to single strand DNA (ssRNA) or double strand DNA (dsDNA).

In a specific embodiment, a composition of the medium may include the substance increasing Rad51 activity at a concentration of 0.1 μM to 50 μM in a basic medium. Specifically, the basic medium may be a basic medium used for culturing mammalian eggs. The basic medium may vary depending on the species of the mammal, but may include any one or more selected from the group consisting of inorganic salts, carbon sources, amino acids, bovine serum albumin, and cofactors. The basic medium may include all common media known to those skilled in the art. NaCl, KCl, NaHCO₃, etc. may be used as the inorganic salts, glucose, sodium pyruvate, calcium lactate, etc. may be used as the carbon sources, essential amino acids and non-essential amino acids including glutamine may be used as the amino acids, and other trace elements, buffers, etc. may be used as the cofactors. The medium may be, for example, selected from the group consisting of Minimal Essential Medium (MEM), Dulbecco modified Eagle Medium (DMEM), Roswell Park Memorial Institute Medium (RPMI), Keratinocyte Serum Free Medium (K-SFM), Iscove's Modified Dulbecco's Medium (IMDM), F12, and DMEM/F12. Further, the medium may include a neutral buffer (e.g., phosphate and/or high concentration bicarbonate) and protein nutrients (e.g., serum, such as FBS, fetal calf serum (FCS), horse serum, serum replacement, albumin, or essential amino acids and non-essential amino acids, such as glutamine and L-glutamine) in an isotonic solution. Furthermore, the medium may include lipids (fatty acid, cholesterol, serum HDL or LDL extract) and other components found in most types of storage media thereof (e.g. transferrin, nucleoside or nucleotide, pyruvate, any ionized form or salt sugars, such as glucose, glucocorticoids such as hydrocortisone, and/or reducing agents such as β-mercaptoethanol). The basic medium may also include antibiotics. The substance increasing Rad51 activity may be included at a concentration of 0.1 μM to 50 μM, 0.1 μM to 40 μM, 0.1 μM to 30 μM, 1 μM to 50 μM, 1 μM to 30 μM, 5 μM to 30 μM or 5 μM to 25 μM in the basic medium. In this regard, when the concentration of the substance increasing Rad51 activity included in the basic medium is less than the above range, reactive oxygen species may not be effectively removed, and it deteriorates blastocyst development efficiency of an egg, and thus there is a problem in that a good-quality egg may not be obtained. When the concentration is more than the above range, the substance increasing Rad51 activity acts on an egg for a long time, and thus there is a problem in that the substance prevents the egg from maturing.

In a specific embodiment, the composition may further include an agent reducing H3K9me3 methylation. The agent reducing methylation may be an agent increasing expression of a member of KDM4 family of histone demethylases. For example, the agent may be an agent increasing expression or activity of KDM4A(JMJD2A), KDM4B(JMJD2B), KDM4C(JMJD2C), KDM4D(JMJD2D), or a combination thereof.

Another aspect provides method of improving embryonic development efficiency of an egg, the method including culturing the egg in a medium including the substance increasing Rad51 activity. A detailed description of the medium including the substance increasing Rad51 activity is the same as described above.

In a specific embodiment, the method may include contacting the egg with an agent reducing H3K9me3 methylation. A detailed description of the agent reducing H3K9me3 methylation is the same as described above. Further, the egg may be frozen and then thawed.

The method may include culturing the egg for 1 day to 10 days in a medium including the substance increasing Rad51 activity. Specifically, culturing may be performed for 1 day to 10 days, 1 day to 8 days, 1 day to 7 days, 2 days to 8 days, 2 days to 6 days, or 3 days to 6 days. In this regard, when the culture period is less than the above range, there is a problem in that an embryo does not sufficiently develop into a blastocyst, and when the culture period is more than the above range, there is a problem in that it is difficult to obtain a qualitatively improved blastocyst due to excessive maturation of the egg.

In addition, the method may further include developing the embryo obtained in the culturing into a subject. The subject may be a morula, a blastula, and/or a gastrula.

Still another aspect provides an embryo, a blastocyst, and/or an embryonic stem cell, which is prepared by the above method. Still another aspect provides a composition for implanting, the composition including, as an active ingredient, the embryo and/or the blastocyst prepared by the above method.

Still another aspect provides a method of increasing somatic cell nuclear transfer efficiency using the substance increasing Rad51 activity. The efficiency may be a success rate of somatic cell nuclear transfer or an embryonic development rate of cells produced by the somatic cell nuclear transfer. The embryonic development rate may refer to development of an embryo into a blastocyst through a 2-cell stage, a 4-cell stage, and an 8-cell stage.

The present inventors found that Rad51 expression is very low during a cloning process of cells produced by somatic cell nuclear transfer, as compared with in vitro fertilization process, and they confirmed a result that the substance increasing Rad51 activity dramatically improves efficiency of the somatic cell nuclear transfer. Therefore, Rad51 activator may be usefully applied to cell preparation by somatic cell nuclear transfer, and efficiency of the somatic cell nuclear transfer may be remarkably improved by screening for the substance increasing Rad51 activity.

The method may include preparing a nucleus of a somatic cell and an enucleated egg; culturing the nucleus of the somatic cell with the enucleated egg in the presence of the substance increasing Rad51 activity; and obtaining a somatic cell nuclear transfer (SCNT) cell.

The method may further include removing a nucleus from an oocyte, implanting one or more nuclei (donor nuclei) of somatic cells, activating the reconstructed nuclear transfer oocyte (embryo), and additionally culturing the oocyte into a blastocyst. The procedures for such somatic cell nuclear transfer (SCNT) may be appropriately modified and carried out by those skilled in the art according to a method disclosed in a literature (Nature 419, 583-587, 10 Oct. 2002), etc.

The method may include adding one or more nuclei of one or more somatic cells, i.e., donor cells, including directly injecting the donor nuclei into the oocyte, or fusing the nuclei with the cell through electrical stimulation.

The “somatic cell nuclear transfer” or “somatic cell nuclear implant” refers to a technology of implanting a nucleus collected from a donor cell into an enucleated recipient cell, and generating a cell genetically identical to the donor cell.

The enucleated egg may be a cumulus-oocyte complex collected from a follicle of a subject, or a commercially available one. The subject may be a mammal including a human. For example, the mammal may include rats, pigs, sheep, dogs, cows, horses, goats, etc. In addition, the enucleated egg may be frozen and/or cryopreserved. The freezing method may include a slow freezing method or a vitrification method which is a flash freezing method.

The method according to a specific embodiment may include forming a blastocyst by in-vitro culturing a nucleus of a somatic cell with an enucleated egg.

The substance increasing Rad51 activity may be added to the medium for culturing the nucleus of the somatic cell with the enucleated oocyte, for example, the substance increasing Rad51 activity may be present in the medium before or after production of SCNT cells, at a 2-cell stage, a 4-cell stage, or a 8-cell stage, or at the time of blastocyst formation. The concentration of the substance increasing Rad51 activity may be appropriately adjusted by those skilled in the art according to the degree of Rad51 activation of the substance or cytotoxicity thereof, for example, the substance may be present at a concentration of 0.1 μM to 50 μM, 0.1 μM to 40 μM, 0.1 μM to 30 μM, 1 μM to 50 μM, 1 μM to 30 μM, 5 μM to 30 μM, or 5 μM to 25 μM in the medium.

The method according to a specific embodiment may include contacting with an agent reducing H3K9me3 methylation. The agent reducing H3K9me3 methylation may be an agent increasing expression of a member of KDM4 family of histone demethylases. The agent may be, for example, an agent increasing expression or activity of KDM4A(JMJD2A), KDM4B(JMJD2B), KDM4C(JMJD2C), KDM4D(JMJD2D), or a combination thereof. The contacting may be performed for the nuclear transfer embryo after fusing the nucleus of the somatic cell with the enucleated egg. Specifically, the embryo may be an embryo before gene activation of the SCNT oocyte begins. For example, the contacting may be contacting the embryo with one or more members of KDM4 family of histone demethylases at 5 hours (5 hpa), between 10 hpa to 12 hpa (i.e., at a 1-cell stage), at about 20 hpa (i.e., at an early 2-cell stage), or between 20 hpa to 28 hpa (i.e., at a 2-cell stage) post activation.

Further, the contacting or injecting may be contacting or injecting one or more members of KDM4 family of histone demethylases with/into the nucleus or cytoplasm of a donor cell, for example, a terminally differentiated somatic cell, before injecting the nucleus of the donor cell into the enucleated oocyte. The contacting or injecting may be contacting with the donor somatic cell for 1 hour or more, or 2 hours or more, and the contacting may be performed for 1 day (24 hours) or more, 2 days or more, 3 days or more, or more than 3 days before nuclear transfer from the donor somatic cell to the enucleated oocyte.

Accordingly, by contacting the nucleus of the somatic cell and the enucleated egg with the agent reducing H3K9me3 methylation, the activity of the histone methylase that suppresses replication in the SCNT embryo may be reduced, and expression of genes related to embryonic development may be enhanced, thereby improving generation and development efficiency of a blastocyst. For example, the development efficiency may be % development of the SCNT embryo into a 2-cell stage, 4-cell stage and 8-cell stage, or a blastocyst stage. In other words, pre-implantation development efficiency of the SCNT embryo into the 8-cell stage or the blastocyst stage may be increased. In one embodiment, changes in the status of the embryo and the number of blastocyst were examined according to the presence or absence of the substance increasing Rad51 activity during the SCNT procedure. As a result, it was confirmed that the number of embryos at the 2-cell stage block was reduced by treatment with the substance increasing Rad51 activity, and the embryonic development rate was significantly increased (FIG. 3A). Therefore, the substance increasing Rad51 activity may overcome a phenomenon of the 2-cell stage block, and may maintain embryonic development, thereby increasing blastocyst development efficiency.

In one embodiment, the method results in an about 5% or more, about 10% or more, about 13% or more, about 15% or more, about 30% or more, about 50% or more, 50% to 80%, or more than 80% increase in the nuclear transfer efficiency, as compared with the SCNT which was performed in the absence of the substance increasing Rad51 activity. In other words, the method increases the efficiency of pre-implantation development of SCNT embryos, the development of embryos to the blastocyst stage, or the development of embryos to the expanded blastocyst stage, whereby about 5%, about 7%, about 10%, about 12 or more, or more than 12% develop to the expanded blastocyst stage. In another embodiment, the method increases 3-fold or more, 4-fold or more, 5-fold or more, 6-fold or more, 7-fold or more, 8-fold or more, or more than 8-fold in the successful development to the blastocyst stage, as compared with the SCNT which was performed in the absence of the substance increasing Rad51 activity. The increase in the SCNT efficiency refers to an increase in the generation or yield of blastocysts. The increase in the generation or yield of blastocysts may be about 110% or more, about 120% or more, about 130% or more, about 140% or more, about 150% or more, or more than about 150% increase, as compared with the SCNT which was performed in the absence of the substance increasing Rad51 activity.

As described above, the method according to an aspect may prevent cell damage of SCNT eggs to improve quality of the eggs by culturing the SCNT eggs in a medium including the substance increasing Rad51 activity. Further, cell death of frozen and thawed SCNT eggs may be reduced by Rad51 activator, thereby enhancing blastocyst formation and production efficiency. Further, since an implantation rate of SCNT eggs is improved, it is possible to produce endangered animals through in vitro fertilization, and since induction of embryonic stem cells is enhanced, embryonic stem cell lines may be efficiently produced.

Still another aspect provides a SCNT embryo, a blastocyst, an embryonic stem cell, or a cloned germ cell which is prepared according to the above method. The embryo is genetically modified. For example, one or more transgenes may be modified in the genetic material of the donor nucleus prior to the SCNT procedure (i.e., prior to collecting the donor nucleus and fusing with the cytoplasm of the recipient oocyte). In a specific embodiment, the embryo may include nuclear DNA derived from the donor somatic cell, cytoplasm derived from the recipient oocyte, and mitochondrial DNA derived from a third donor subject. The embryonic stem cell may be formed by isolating a cell from an inner cell mass in the blastocyst prepared by the above method; and culturing the undifferentiated inner cell mass-derived cell. Further, the embryonic stem cell may be a pluripotent stem cell or a totipotent stem cell.

Still another aspect provides a method of screening for a substance increasing somatic cell cloning efficiency by increasing Rad51 activity, the method including preparing a nucleus of a somatic cell and an enucleated egg; culturing the nucleus of the somatic cell with the enucleated egg in the presence of a candidate; and assessing Rad51 activity of the SCNT cell developed after the culturing.

Cells derived from the SCNT embryo or blastocyst, e.g., embryonic stem cells may be used in a test to determine whether an agent affects differentiation or cell proliferation. For example, ability of the cells to differentiate or proliferate is assessed in the presence or absence of the agent, and thus the cells derived from the SCNT embryo or blastocyst may also be used in the screening to select agents that affect them. The test compound may be any compound of interest, including chemical compounds, small molecules, polypeptides, or other biological agents (e.g., antibodies, cytokines, etc.).

In the method according to a specific embodiment, the assessing of the Rad51 activity may be performed to examine Rad51 expression, which may be performed by using a technology known in the art, such as RT-PCR, an immunostaining method, etc. Further, the assessing of the Rad51 activity may be performed, together with assessing of somatic cell cloning efficiency. When the number of SCNT oocytes, cells at a 2-cell stage, cells at a 4-cell stage, or cells at a blastocyst stage in the culture is changed after addition of the candidate, as compared with prior to addition of the candidate, the candidate may be determined as a substance increasing Rad51 activity and increasing somatic cell cloning efficiency by SCNT.

Still another aspect provides a method of preparing a SCNT egg, the method including preparing a nucleus of a somatic cell and an enucleated egg; culturing the nucleus of the somatic cell with the enucleated egg in a medium including the substance increasing Rad51 activity to obtain a SCNT egg; and performing in vitro fertilization of the SCNT egg with liquid semen. A detailed description of the method of culturing the nucleus of the somatic cell and the enucleated egg in the medium including Rad51 activator is the same as described above. The SCNT cell may be an SCNT egg, specifically, an embryo at the 2-cell stage, 4-cell stage, or 8-cell stage, wherein the embryo may develop to reach the blastocyst stage.

The method according to a specific embodiment includes performing in vitro fertilization of the SCNT egg with liquid semen. The semen may be semen collected from the vas deferens of a subject. The subject may be a mammal including a human. For example, the mammal may include rats, pigs, sheep, dogs, cows, horses, goats, etc. The in vitro fertilization of the SCNT cell and liquid semen may be performed in a medium composition for in vitro fertilization for 1 day to 7 days. In this regard, when the in vitro fertilization period is less than the above range, there is a problem in that fertilization does not occur, and when the period is more than the above range, there is a problem in that the embryo degenerates. In the above procedure, contacting with or injecting the substance increasing Rad51 activity may be further included. A detailed description of the substance increasing Rad51 activity is the same as described above. The substance increasing Rad51 activity may be supplemented with or injected into the IVF egg, after injection of semen into the SCNT egg, before formation of pronucleus (within 18 hr after semen injection), specifically, after injection of semen and before the 2-cell stage. Therefore, the SCNT egg may increase production efficiency, since the SCNT egg (embryo) efficiently develops without development block, and successfully develops into a blastocyst through 2-, 4- and 8-cell stages without developmental defects or loss of viability.

### Advantageous Effects of Disclosure

The present disclosure relates to a composition for embryonic development, the composition including a Rad51 activator, and a method of improving an embryonic development rate using the same. The cell efficiently develops into a blastocyst without cell damage and/or development block, and therefore, somatic cell-cloned egg may be produced using in vitro fertilization procedures.

### BRIEF DESCRIPTION OF DRAWINGS

FIG. 1A shows RT-PCR results of analyzing Rad51 expression in in vitro fertilization (IVF), somatic cell nuclear transfer (SCNT) by using fresh eggs, and SCNT by injecting Kdm4a mRNA into eggs;

FIG. 1B shows RT-PCR results of analyzing Rad51 expression in IVF, SCNT by using frozen eggs, and SCNT by using fresh eggs;

FIG. 2 shows immunostaining results of analyzing Rad51 expression in IVF, SCNT by using frozen eggs, and SCNT by using fresh eggs;

FIG. 3A shows photographs showing changes in the number of embryos at 2-cell stage block and blastocysts according to the presence or absence of Rad51 activator during SCNT;

FIG. 3B shows photographs showing a relationship between autophagy and mitochondria in IVF eggs, SCNT eggs, and SCNT+RS-1 eggs;

FIG. 4 shows photographs showing mitochondrial activity in IVF eggs, SCNT eggs, and SCNT+Kdm4a eggs;

FIG. 5A shows photographs showing cell damage in IVF eggs, SCNT eggs, and SCNT+RS-1 eggs;

FIG. 5B shows photographs showing DNA damage biomarker expression in IVF eggs, SCNT eggs, and SCNT+RS-1 eggs;

FIG. 5C shows photographs showing DNA damage in the different phases of the cell cycle in IVF eggs, SCNT eggs, and SCNT+RS-1 eggs;

FIG. 6 shows a graph showing DNA breaks in IVF eggs, SCNT eggs, and SCNT+RS-1 eggs;

FIG. 7 shows a graph showing an increase or a decrease in gene expression to examine the effect of RS-1 treatment on SCNT eggs;

FIG. 8 shows photographs of H3K9me3 staining to examine SCNT mechanism patterns according to RS-1 treatment and Kdm4a mRNA treatment;

FIG. 9A shows a table showing a production of cloned pups rate after SCNT according to RS-1 treatment; and

FIG. 9B shows a graph showing an SCNT-PSCs derivation rate of SCNT eggs according to RS-1 treatment.

### MODE OF DISCLOSURE

Hereinafter, the present disclosure will be described in more detail with reference to exemplary embodiments. However, these exemplary embodiments are only for illustrating the present disclosure, and the scope of the present disclosure is not limited thereto.

### Example 1. Somatic Cell Nuclear Transfer (SCNT)

5IU PMSG and 5IU hCG hormone were administered into 8-week to 10-week-old female BDF/1 mice. 14 hr after hCG administration, cumulus cells were isolated from superovulated mice using hyaluronidase and eggs were collected. The isolated cumulus cells were refrigerated for use as somatic cell for donor cells, and the isolated eggs were stored in a KSOM culture medium in an incubator at 37° C. until experiments started. Thereafter, to carry out a somatic cell cloning experiment, nuclei were removed from matured eggs, and cumulus cells isolated in advance were directly injected as donor cells. The cumulus cells injected as donor cells were artificially activated in a culture medium containing a Rad51-stimulatory compound 1 (RS-1) for 6 hr. Thereafter, the cells were incubated in the culture medium containing RS-1 reagent for 22 hr. 22 hr later, the cells were incubated in a KSOM culture medium for 72 hr to 96 hr.

### Example 2. Preparation of Kdm4a mRNA and Injection of Kdm4a mRNA into Somatic Cell Cloned Egg

2-1. Preparation of Kdm4a mRNA

Full-length mouse Kdm4a/Jhdm3a cDNA was cloned into a pcDNA3.1 plasmid containing poly(A)83 at the 3′-end of cloning site using an In-Fusion kit (Clonetech #638909). mRNA was synthesized from the linearized template plasmid by in vitro transcription using a mMESSAGE mMACHINE T7 Ultra Kit (Life Technologies #AM1345). The synthesized mRNA was dissolved in nuclease-free water. The concentration of mRNA was measured by a NanoDrop ND-1000 spectrophotometer (NanoDrop Technologies); Aliquots of mRNA were stored at −80° C. until use.

2-2. Injection of Kdm4a mRNA into Somatic Cell Cloned Egg

2 μg/μl of Kdm4a mRNA prepared in Example 2-1 was injected into a cloned oocyte treated with RS-1 for 6 hr, together with ˜10 pl of a control, and a cloned oocyte not treated with RS-1 in an activated culture medium using a piezo-driven micromanipulator, respectively. After injection, the oocytes were cultured for 22 hr in an RS-1-added KSOM culture medium and a non-RS-1 added culture medium, respectively and then cultured for 72 hr to 96 hr in the non-RS-1 added culture medium. Then, blastocyst efficiency was observed.

### Example 3. Comparative Analysis of Rad51 Expression in Somatic Cell Nuclear Transfer Egg Relative to In Vitro Fertilized Egg

Expression of Rad51 which regulates homologous recombination was compared and analyzed by qRT-PCR in in-vitro fertilized (IVF) eggs, somatic cell nuclear transfer eggs (SCNT, F/T SCNT) of Example 1, and cloned eggs (SCNT+Kdm4a) injected with Kdm4a mRNA during SCNT of Example 2. In detail, mRNAs of the embryos were isolated using a Dynabeads mRNA DIRECT kit (Dynal Asa, Oslo, Norway). About 20 embryos at a pronuclear stage (PN), 2-cell stage, or 4-cell stage were washed with a lysis/binding buffer, and then mixed with Dynabeads oligo dT₂₅, and bound therewith at room temperature. mRNA were bound with beads, and washed with Buffer-A twice, and then additionally washed with Buffer-B. Thereafter, a Tris-HCl solution was added, and mRNA was separated from beads at 73° C. cDNA was synthesized from the separated mRNA using oligo dT primers. The synthesized cDNA was used as a template to analyze mouse Rad51 expression by qRT-PCR. Nucleotide sequences of the primers used herein are as follows:

Forward primer:
5′-AGCTTTCAGCCAGGCAAAT-3′,
Reverse primer:
5′-GCTTCAGCTTCAGGAAGACA-3′.

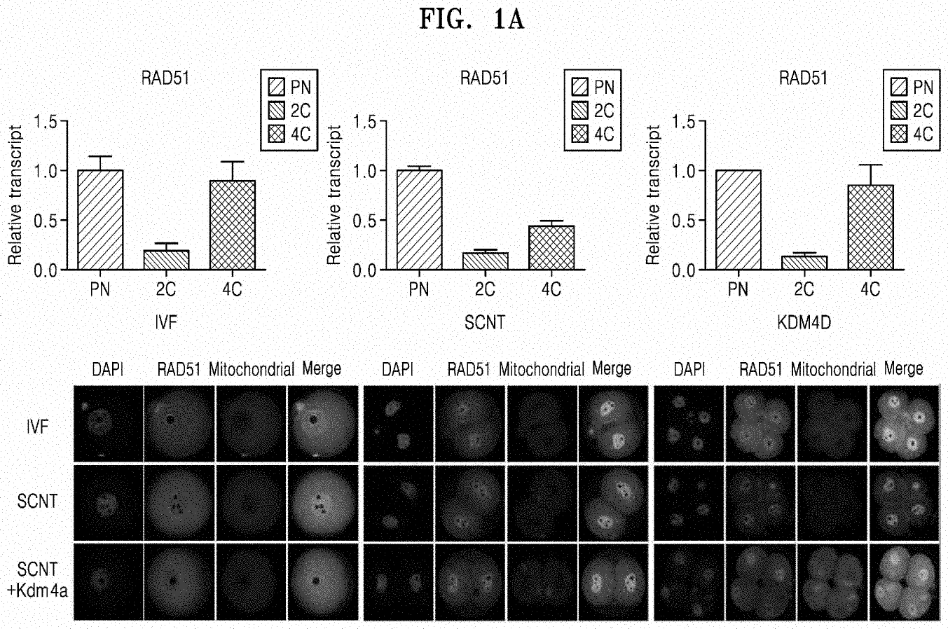

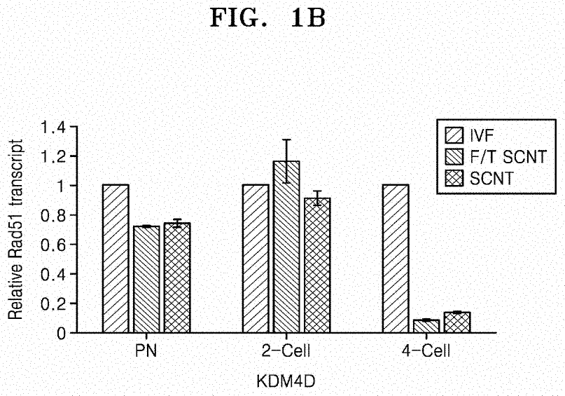

As a result of comparative analysis of Rad51 expression in the embryos at the PN stage, 2-cell stage, or 4-cell stage, IVF showed very abundant expression of Rad51 at the 1-cell stage, but showed a significant decreasing tendency at the 2-cell stage, and an increasing tendency at the 4-cell stage, similarly to the 1-cell stage. In contrast, SCNT showed similar expression patterns to IVF at the 1-cell stage and 2-cell stage. However, SCNT at the 4-cell stage did not show the increased expression similar to the 1-cell stage, as compared with IVF. However, SCNT+Kdm4a showed similar expression patterns to IVF (FIG. 1A).

Rad51 expression was remarkably low at the 4-cell stage formed by SCNT using fresh eggs and frozen eggs, as compared with that at 4-cell stage formed by IVF (FIG. 1B).

Next, an immunostaining method was used to reanalyze the result of remarkably low RNA expression of Rad51 at the 4-cell stage formed by SCNT, as compared with that at 4-cell stage formed by IVF. In detail, 30 hr after activating the somatic cell cloned eggs, the eggs at the 2-cell stage were washed with 0.1% BSA-added PBS, and then treated with 4% paraformaldehyde at room temperature for 30 min. After treatment with 0.1% Triton X-added PBS/0.1% BSA for 24 hr, Rad 51, γH2AX, Mitotracker, and LC3B antibodies were treated at room temperature for 2 hr. After washing with 0.1% BSA-added PBS for 10 min three times, goat anti-mouse antibodies and donkey anti-rabbit antibodies were treated for 1 hr. After washing with PBS/0.1% BSA for 10 min three times, nuclei were stained with 4′, 6-diamidino-2-phenylindole (DAPI). The stained eggs were fixed on a slide glass, and observed and analyzed by confocal fluorescence microscopy.

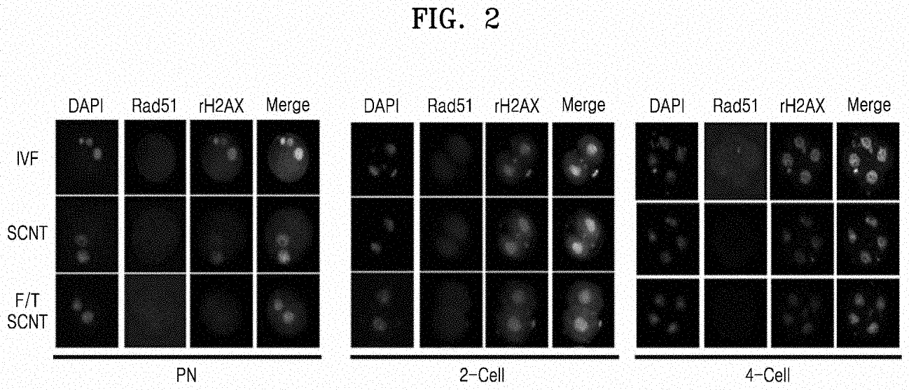

As a result, there was no difference in the Rad51 protein quantity between IVF, SCNT, and SCNT by using frozen eggs until the 2-cell stage, but significantly low Rad51 protein quantity was observed at the 4-cell stage formed by SCNT (FIG. 2).

### Example 4. Examination of Somatic Cell Cloning Efficiency Improvement by Treatment with Compound Increasing Rad51 Activity and Examination of Biomarkers

4-1. Examination of Somatic Cell Cloning Efficiency Improvement

Based on the results of Example 3, RS-1 which is a substance increasing Rad51 activity was treated during somatic cell cloning procedures, and then whether somatic cell cloning efficiency was improved or not was examined. First, to examine whether RS-1 induced toxicity to egg development, IVF eggs and SCNT eggs (F/T SCNT) of Example 1 were treated with 0 μM, 1 μM, 5 μM, and 10 μM of RS-1, respectively, and an appropriate concentration was selected.

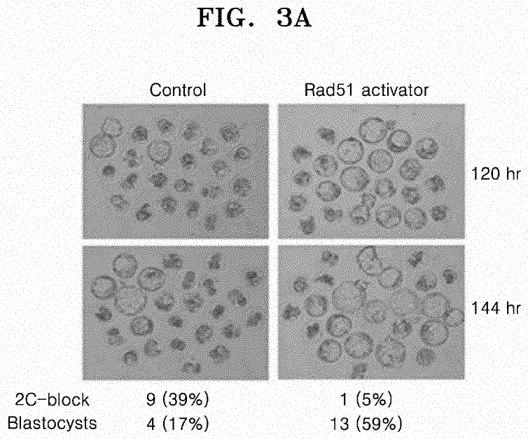

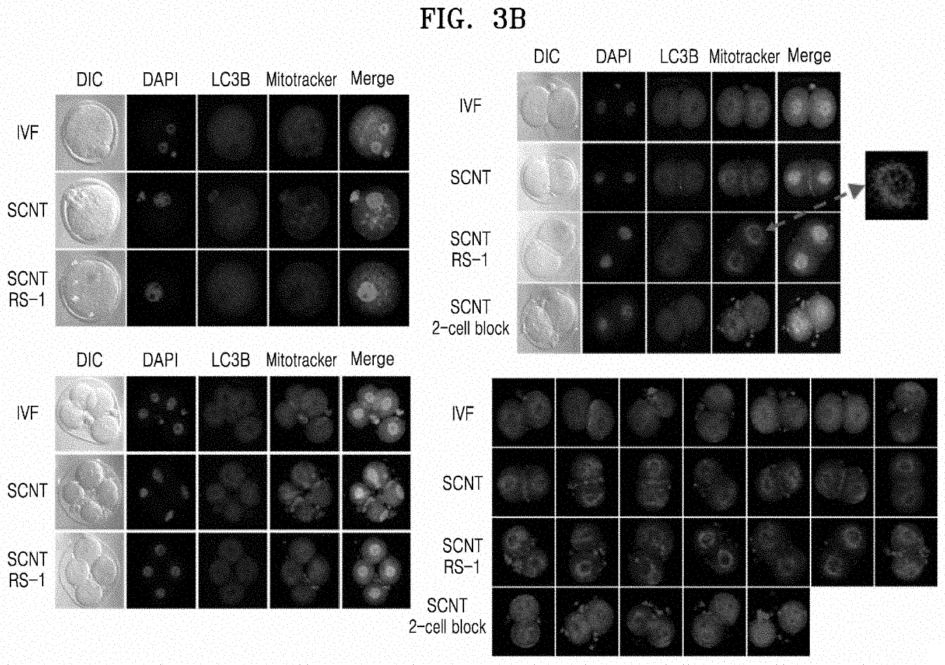

As a result, it was confirmed that embryonic development rates were significantly increased at 10 μM (Table 1) (control group: 31%, RS-1: 76%). As expected, it was also confirmed that the number of embryos at the 2-cell stage block was reduced by treatment with RS-1 (control group: 39%, RS-1 5%), and the embryonic development rate was also significantly increased (control group: 17%, RS-1 59%) (FIG. 3A).

TABLE 1

| | | | Number of | Number of | | | | |
|---|---|---|---|---|---|---|---|---|
| | | | embryos | embryos | Number | | | |
| | Concen- | Number | at 2-cell | at 4-cell | of embryos | | Number of | |
| | tration | of NT | stage | stage | at 2-cell stage | | blastocysts | |
| Treatment | (μM) | oocytes | (%)+ | (%)# | block (%)# | | (%)# | |
| Control | — | 48 | 42 | 30 | 12 | (28.6) | 10 | (23.8) |
| group | | | | | | | | |
| RS-1 | 1 | 45 | 42 | 35 | 7 | (16.7) | 8 | (19) |
| RS-1 | 5 | 46 | 43 | 36 | 7 | (16.3) | 18 | (41.9) |
| RS-1 | 10 | 54 | 50 | 47 | 3 | (6) | 28 | (56) |

Control group: Control oocyte.
+Based on the number of reconstructed oocytes
#Based on the number of embryos at 2-cell stage

4-2. Biomarker Expression and Examination of Relationship

A relationship between autophagy and mitochondria was examined at 1-cell stage, 2-cell stage, and 4-cell stage of IVF eggs, SCNT eggs (F/T SCNT) of Example 1, and RS-1-treated SCNT eggs (SCNT+RS-1) of Example 4-1 by staining with LC3B which is an autophagy marker and mitotracker which is a mitochondrial marker, respectively. In detail, to evaluate mitochondria distribution, SCNT-derived somatic cell cloned eggs at the 1-cell stage, 2-cell stage, and 4-cell stage were stained using MitoTracker Orange CMTMRos (Molecular Probes). Mitotracker was used at a concentration of 300 nM in an M16 culture medium supplemented with 0.3% BSA at 37° C. for 30 min in the dark. After washing, the oocytes were fixed and immunofluorescently stained with LC3B antibody and then nuclei were stained with DAPI.

As a result, at the 1-cell stage, only the SCNT group showed unique small dots in the cytoplasm, resulting from autophagy and mitochondria condensation, and RS-1-treated SCNT group showed a similar expression pattern to IVF group. At the 2-cell stage, IVF group showed uniform distribution of autophagy and mitochondria expression in the nucleus and cytoplasm, whereas SCNT group and SCNT+RS-1 group showed intensive autophagy and mitochondria expression in the nucleus. In particular, SCNT group at the 2-cell stage block showed characteristics of expression only in the cytoplasm without expression in the nucleus. At the 4-cell stage, all groups showed similar expression patterns (FIG. 3B).

### Example 5. Analysis of Mitochondrial Activity in SCNT Eggs Relative to IVF Eggs

To examine mitochondrial activity and potential in IVF eggs, SCNT eggs (F/T SCNT) of Example 1, and cloned eggs (SCNT+RS-1) injected with Kdm4a mRNA during SCNT of Example 2, each egg was stained with JC-1. In detail, to measure mitochondrial activity in SCNT-derived somatic cell cloned eggs at the 1-cell stage, 2-cell stage, and 4-cell stage, JC-1 (Thermo Fisher Scientific, Waltham, Mass., USA) was added at a concentration of 1 pg/ml to a culture medium, followed by incubation for 20 min in the dark. Thereafter, nuclei were stained with Hoechst (Sigma).

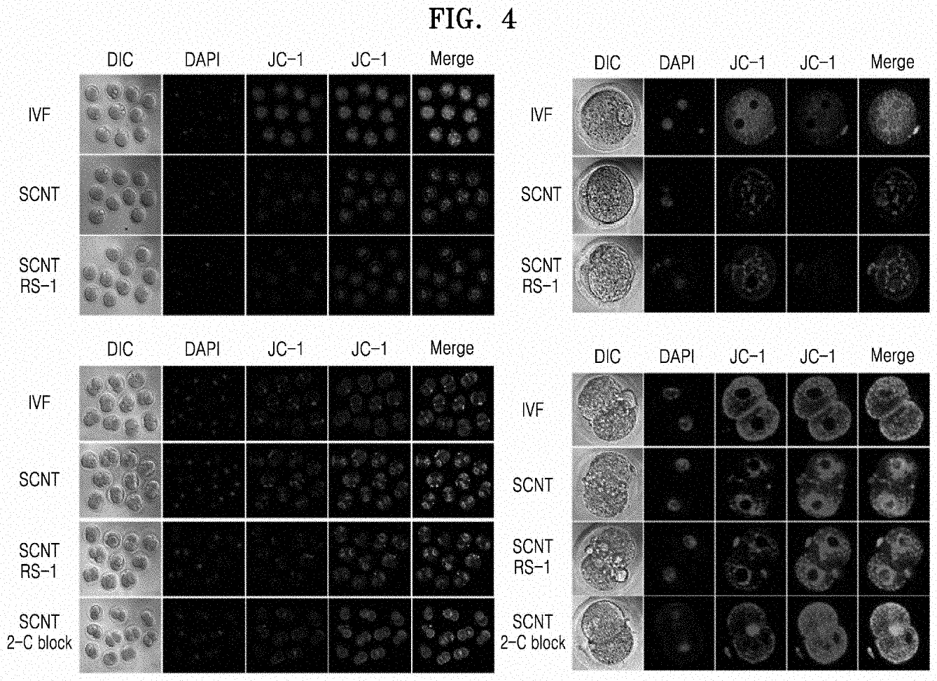

As a result, it was confirmed that the IVF group at the 2-cell stage showed abundant expression of mitochondria around the cytoplasm and nucleus. In contrast, it was confirmed that the SCNT group and the SCNT+Kdm4a group showed unstable expression of mitochondria due to partial condensation in the cytoplasm. It was also confirmed that the SCNT group and the SCNT+Kdm4a group at the 4-cell stage showed relative expression of mitochondria only on the surface of cytoplasm. Therefore, the results indicate that the IVF group and the SCNT group showed different mitochondria expression patterns from each other (FIG. 4).

### Example 6. Examination of Cell Damage and DNA Damage According to RS-1 Treatment

6-1. Examination of Cell Damage

To examine cell damage of somatic cell cloned eggs according to RS-1 treatment, generation of reactive oxygen species was examined in IVF eggs, SCNT eggs (F/T SCNT) of Example 1, and RS-1-treated SCNT eggs (SCNT+RS-1) of Example 4-1 at the 2-cell stage. In detail, IVF group, SCNT+RS-1 group, and SCNT group at the 2-cell stage were incubated at 37° C. for 30 min in the dark in a culture medium supplemented with 5 μM of CellROX oxidative stress reagent for 30 min. Thereafter, each group was washed with 0.1% PVA-added D-PBS, and nuclei were stained with Hoechst (Sigma).

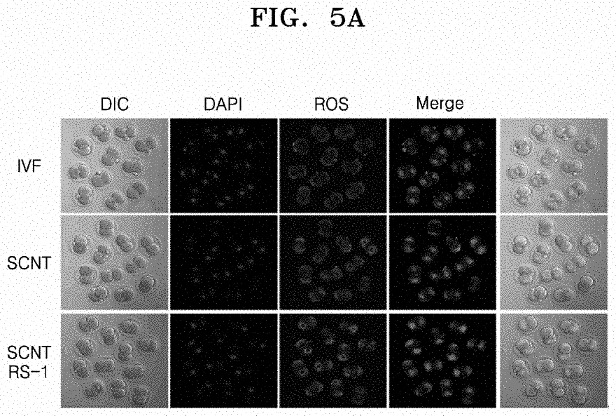

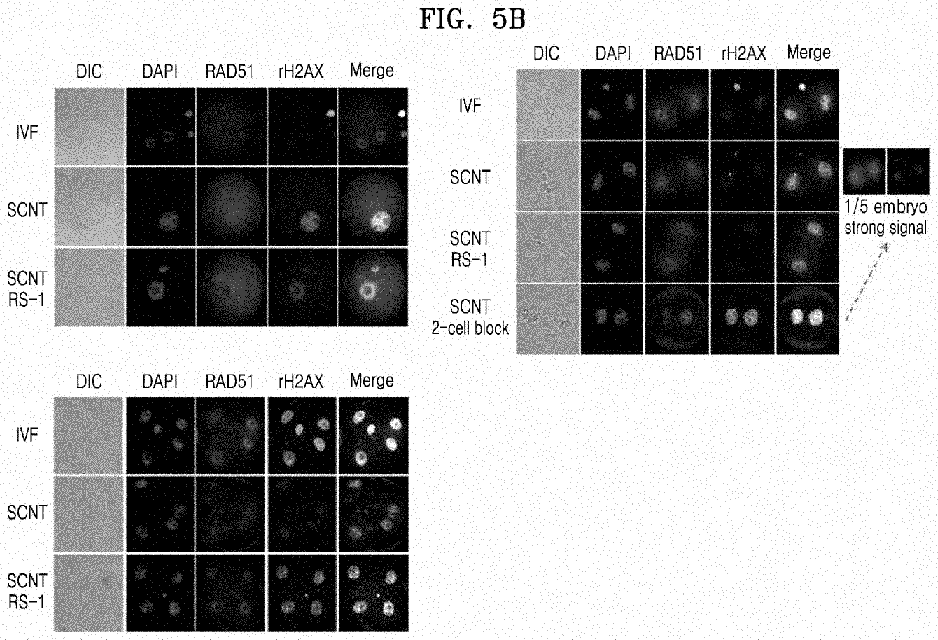

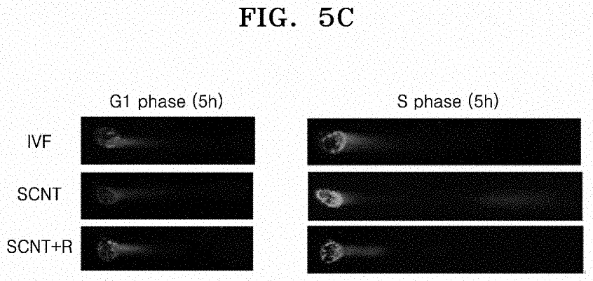

As a result, reactive oxygen species (ROS) tended to decrease on the whole in IVF group. In contrast, reactive oxygen species tended to increase in the nuclei of SCNT+RS-1 group and SCNT group. (FIG. 5A).

6-2. Examination of DNA Damage

To examine DNA damage of somatic cell cloned eggs according to RS-1 treatment, expression of a DNA damage biomarker (rH2AX) was examined in IVF eggs, SCNT eggs (F/T SCNT) of Example 1, and RS-1-treated SCNT eggs (SCNT+RS-1) of Example 4-1. In detail, IVF group, SCNT group, and SCNT+RS-1 group fixed in 4% paraformaldehyde were arrested at the 1-cell stage, 2-cell stage, and 4-cell stage, and then incubated with rH2AX (Abcam, ab22551) Rad51 (Abcam, ab63801) for 2 hr, followed by incubation in the presence of goat anti-mouse antibody diluted with PBS/0.1% BSA at 1:200, and goat anti-rabbit antibody at the same concentration for 1 hr. Thereafter, nuclei were stained with DAPI.

As a result, it was confirmed that IVF group at the 1-cell stage showed very poor rH2AX and Rad51 expression. In contrast, the SCNT group showed highly expression of Rad51 and rH2AX protein in the nucleus, and SCNT+RS-1 group showed a significant reduction in DNA damage. In addition, SCNT+RS-1 group at the 2-cell stage block showed very strong DNA damage in the nucleus, as compared with other groups. Interestingly, IVF group at the 4-cell stage showed very strong Rad51 and rH2AX expression in the nucleus, and SCNT+RS-1 group at the 4-cell stage also showed similar Rad51 and rH2AXIVF expression in the nucleus. In contrast, SCNT group at the 4-cell stage showed very poor Rad51 and rH2AX expression in the nucleus. In other words, when RS-1 treatment was performed during SCNT procedures, DNA damage significantly increased from the 2-cell stage to the 4-cell stage during cloning (FIGS. 5B and 5C).

### Example 7. Examination of DNA Breaks

To examine DNA breaks in IVF eggs, SCNT eggs (F/T SCNT) of Example 1, and RS-1-treated SCNT eggs, a comet assay was performed. In detail, to examine DNA damage, each sample in a 1 ml tube from which a culture medium was removed was suspended in 1% agarose at 37° C., and then fixed on a pre-coated slide (Trevigen). The slide was incubated at 4° C. for 4 hr, and then immersed in a solution (Trevigen) at 4° C. for 4 hr. The slide was removed from the lysis solution, and immersed in a 1×TAE buffer at 4° C. for 30 min, followed by electrophoresis for 30 min to 40 min. The slide was immersed in 1 M ammonium acetate at room temperature for 30 min, and then fixed at 75° C. After fixing at room temperature for 30 min, incubation was performed at 42° C. for 20 min until agarose was completely dried. Thereafter, staining was performed using a 1×SYBR green I staining solution for 5 min.

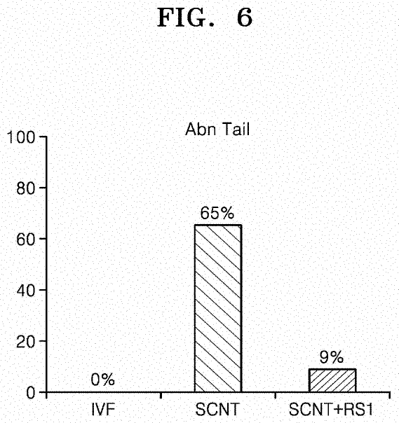

As a result, it was confirmed that DNA breaks frequently occurred as the 3′-end became significantly longer in the SCNT group, as compared with the IVF group. However, it was confirmed that DNA breaks were decreased as the 3′-end became significantly short in the RS-1-treated SCNT+RS-1 group. It was also confirmed that the abnormal 3′-end was generated in the SCNT group. It was also confirmed that generation of the abnormal 3′-end was significantly reduced in the SCNT+RS-1 group (FIG. 6).

### Example 8. Examination of Effect of RS-1 Treatment on SCNT Eggs

To examine the effect of RS-1 treatment on SCNT eggs, RS-1 was treated to cloned eggs at the 2-cell stage during the somatic cell cloning procedure of Example 1, followed by RNA sequencing. At this time, IVF eggs were used as a negative control group. In detail, complementary DNA (cDNA) was amplified using a SMARTer ultra-low input RNA Kit for cDNA preparation (Takara, 634890) according to the manufacturer's instructions. cDNA was fragmented into about 200 bp fragments using a M220 sonicator (Covaris). The fragmented cDNAs were subjected to end-repair, and an adapter was ligated thereto. Sequencing libraries were prepared using a ScriptSeq v2 kit (Illumina) according to the manufacturer's instructions. Single-end sequencing was performed in HiSeq2500 (Illumina), and mapped to the mm9 mouse genome using STAR (v2.5.2b, https://github.com/alexdobin/STAR). Subsequently, kilobase per million read (FPKM) was calculated by cuff link (v2.2.1) with default options.

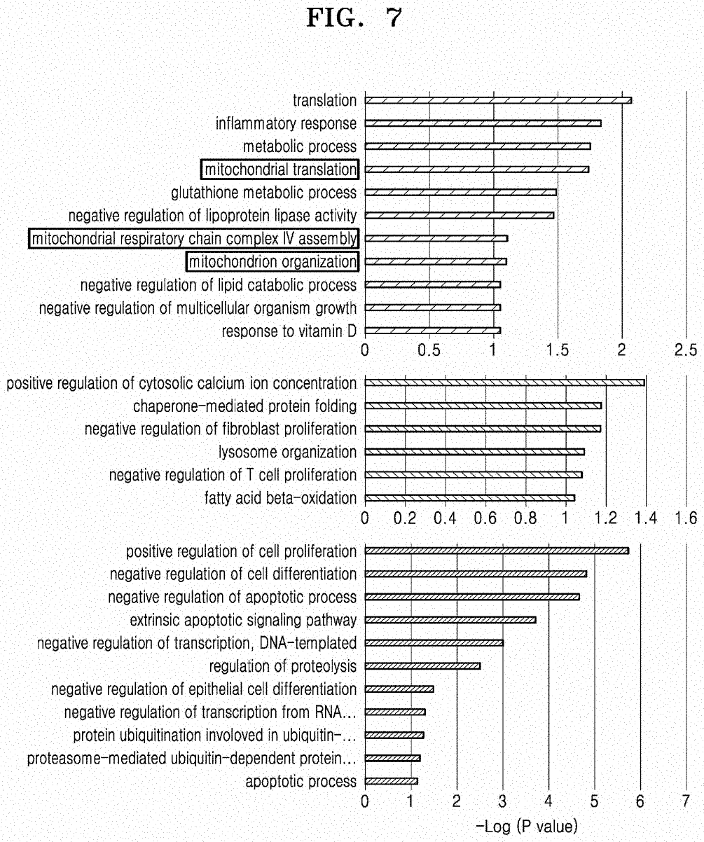

As a result, it was confirmed that expression of genes related to translation, immune response, metabolic process, and mitochondrial translation was increased, and the SCNT+RS-1 group showed a similar expression pattern to the IVF group. It was also confirmed that cell proliferation-related genes were increased and cell differentiation-related genes were decreased (FIG. 7).

### Example 9. Examination of SCNT Efficiency According to RS-1 Treatment and mRNA Injection

To examine cloning mechanisms in the RS-1-treated SCNT egg (SCNT+RS-1) of Example 3 and the cloned egg (SCNT+Kdm4a) injected with Kdm4a mRNA during SCNT of Example 2, genes expressed in each egg were examined in the same manner as in Example 8.

As a result, it was confirmed that 190 genes were significantly increased in the SCNT+RS-1 group and 45 genes (out of 1,314 genes) were significantly increased in the SCNT+Kdm4a group. It was also confirmed that 414 genes were significantly decreased in the SCNT+RS-1 group and 3 genes (out of 478 genes) were significantly decreased in the SCNT+Kdm4a group.

Further, it was demonstrated by H3K9me3 staining that SCNT according to RS-1 treatment and Kdm4a mRNA treatment occurred through different mechanisms. In detail, IVF group, SCNT group, and SCNT+RS-1 group fixed in paraformaldehyde were arrested at the 1-cell stage, and then incubated with H3K9me3 antibody (Millipore, 07-442) for 2 hr, followed by incubation for 1 hr in the presence of goat anti-rabbit antibody diluted with PBS/0.1% BSA at 1:200. Thereafter, nuclei were stained with DAPI.

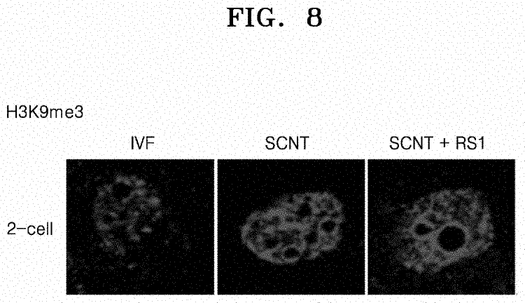

Consequently, it was confirmed that SCNT according to RS-1 treatment and Kdm4a mRNA treatment occurred through the different mechanisms (FIG. 8).

### Example 10. Examination of Cloned Pups Formation Rate and Stem Cell Derivation Rate According to RS-1 Treatment

10-1. Examination of Cloned Pups Formation Rate Through Cloned Mice Production

It is reported that a success rate of cloned mouse production is generally about 1% after normal somatic cell cloning. Therefore, implantation and cloned pups formation rates of mice were examined according to treatment of SCNT eggs with RS-1. In detail, SCNT and SCNT+RS-1 oocytes at the 2-cell stage were implanted into the uterus of surrogate female ICR mice at 0.5 days after pseudopregnancy. At 19.5 days after implantation, the somatic cell cloned mice were separated from the uterus of the surrogate mother, and then the cloned mice were nurtured together with normal ICR mice born on the same day by ICR mice smeared with the smell of the surrogate mother.

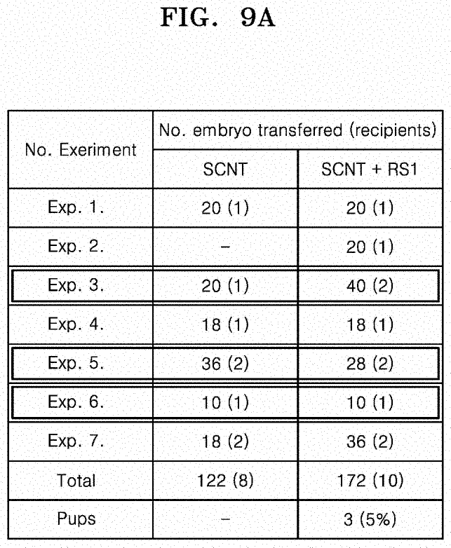

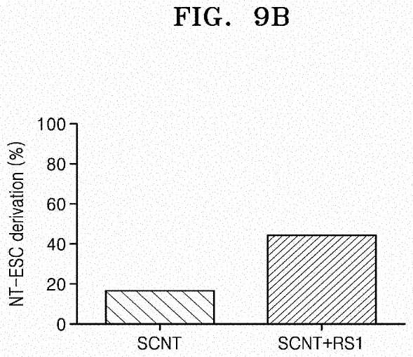

As a result, it was confirmed that the production rate of cloned mouse was increased up to 5% in the RS-1-treated SCNT+RS-1 group. Further, there was no toxicity problem during development. Therefore, there is an advantage that when RS-1 is treated during production of SCNT eggs, cloned mice may be efficiently produced without toxicity problem (FIG. 9A).

10-2. Examination of Stem Cell Establishment Efficiency

To examine stem cell establishment efficiency according to RS-1 treatment, embryonic stem cells were induced from blastocysts of SCNT eggs (SCNT) of Example 1 and RS-1-treated SCNT eggs (SCNT+RS-1) of Example 3. In detail, blastocysts of each group was cultured on mouse embryonic fibroblast (MEF cells) feeder cells in a mouse embryonic stem cell culture medium. 20% KSR, 0.1 mM β-mercaptoethanol, 1% non-essential amino acids, 100 units/ml penicillin, 100 μg/ml streptomycin (all products available from GIBCO), 1.5×10³ units/ml of recombination mLif (Chemicon) were used in the culture medium. Subculture was performed using Trypsin-EDTA, and the established embryonic stem cells of somatic cell cloned mice were treated with alkaline phosphatase. The established stem cells were evaluated by histochemical staining.

As a result, it was confirmed that, as compared with the non-RS-1 treated group (SCNT), the RS-1 treated group (SCNT+RS-1) showed a significantly high induction rate of embryonic stem cells (17% vs. 45%) (FIG. 9B).

1. A composition for increasing efficiency of embryonic development or embryo formation, the composition comprising a substance increasing Rad51 activity.

2. The composition of claim 1, wherein the substance increasing Rad51 activity is a compound of the following Formula 1 or a derivative thereof:

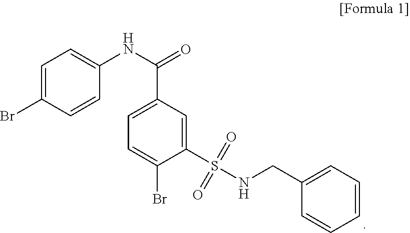 [SMILES: O=C(Nc1ccc(Br)cc1)c1ccc(Br)c(S(=O)(=O)NCc2ccccc2)c1]

3. The composition of claim 1, further comprising an agent reducing H3K9me3 methylation.

4. The composition of claim 3, wherein the agent reducing H3K9me3 methylation is an agent increasing expression of a member of KDM4 family of histone demethylases.

5. The composition of claim 4, wherein the agent is an agent increasing expression or activity of KDM4A(JMJD2A), KDM4B(JMJD2B), KDM4C(JMJD2C), KDM4D(JMJD2D), or a combination thereof.

6. The composition of claim 1, wherein a concentration of the substance increasing Rad51 activity is 5 μM to 15 μM.

7. The composition of claim 1, wherein the substance increasing Rad51 activity is present at a 4-cell stage of the embryo.

8. The composition of claim 1, wherein the embryonic development is development of embryos to blastocysts or formation of blastocysts.

9. The composition of claim 1, wherein the embryo is formed through in-vitro artificial fertilization.

10. The composition of claim 9, wherein in the artificial fertilization, the substance increasing Rad51 activity is present after injection of semen into eggs and before formation of pronucleus.

11. The composition of claim 1, wherein the embryo is formed by implanting a nucleus of a somatic cell into an enucleated egg.

12. A method of increasing efficiency of embryonic development or embryo formation, the method comprising culturing an egg in a medium comprising a substance increasing Rad51 activity.

13. The method of claim 12, further comprising contacting the egg with an agent reducing H3K9me3 methylation.

14. The method of claim 12, wherein the egg is frozen and then thawed.

15. The method of claim 12, wherein, in the embryo obtained by the culturing, damage is induced during DNA replication.

16. The method of claim 12, wherein the damage is induced after a 2-cell stage and before a 4-cell stage.

17. The method of claim 12, wherein the substance increasing Rad51 activity reduces a 2-cell stage block of the embryo obtained by the culturing.

18. The method of claim 12, further comprising developing the embryo obtained by the culturing into a subject.

19. An embryo prepared according to the method of claim 12.

20. A blastocyst prepared according to the method of claim 12.

21. An embryonic stem cell prepared according to the method of claim 12.
